(19) United States
(12) Patent Application Publication
(10) Pub. No.: US 2023/0165506 A1
(43) Pub. Date: Jun. 1, 2023

(54) APPARATUS FOR NON-INVASIVE ACQUISITION OF MATERNAL AND/OR FETAL PHYSIOLOGICAL SIGNALS

(71) Applicant: Marani Health, Inc., Oakdale, MN (US)

(72) Inventors: Ann Holder, Stillwater, MN (US); Kathleen Ann Tune, Minneapolis, MN (US); Christian Larson, Minneapolis, MN (US); Mehdi Shokoueinejad, New Brighton, MN (US); Ognjen Milicevic, Belgrade (RS); Sasa Takov, Belgrade (RS)

(21) Appl. No.: 18/060,252

(22) Filed: Nov. 30, 2022

Related U.S. Application Data
(60) Provisional application No. 63/264,772, filed on Dec. 1, 2021.

(51) Int. Cl. A61B 5/344; A61B 5/00; A61B 5/28; A61B 5/296; A61B 5/256

(52) U.S. Cl. CPC A61B 5/344; A61B 5/6823; A61B 5/6802; A61B 5/28; A61B 5/296; A61B 5/256; A61B 2562/0271; A61B 2562/0219

(57) ABSTRACT

A system for non-invasively acquiring maternal and/or fetal biopotential signals includes a wearable device configured to be worn by a pregnant patient comprising a plurality of electrodes configured to detect maternal and/or fetal biopotential signals associated with the patient and her fetus. The system further includes a computing device configured to receive the maternal and/or fetal biopotential signals from each of a plurality of electrode pairs and select, based on processing the maternal and/or fetal biopotential signals, one of the plurality of electrodes to use as a reference electrode during a monitoring session for the patient.

### CROSS REFERENCE TO RELATED APPLICATIONS

This application claims the benefit of U.S. Provisional Patent Application No. 63/264,772, filed Dec. 1, 2021, which is incorporated by reference herein in its entirety.

### BACKGROUND

Reliable assessment of fetal well-being is a persistent challenge of current fetal monitoring technologies, including the non-invasive cardiotocography (CTG) technologies and invasive fetal scalp electrodes. The poor specificity and reliability of these techniques have the potential to lead to adverse maternal and fetal outcomes, including unnecessary cesarean sections, related post-surgical complication, inaccurate detection of fetal hypoxia and other fetal complications.

### SUMMARY

In one example, the disclosure describes a system for non-invasively acquiring at least one of maternal or fetal biopotential signals comprising: a wearable device configured to be worn by a pregnant patient, wherein the wearable device comprises a plurality of electrodes configured to detect at least one of maternal or fetal biopotential signals associated with the patient and her fetus; and a computing device configured to: receive at least one of maternal or fetal biopotential signal data detected by a plurality of electrode pairs of the wearable device, wherein each electrode pair includes two electrodes of the plurality of electrodes of the wearable device, and wherein a first electrode of each of electrode pair is a measurement electrode and a second electrode of each electrode pair is a reference electrode; select, based on processing the at least one of maternal or fetal biopotential signal data detected by the plurality of electrode pairs of the wearable device, one of the plurality of electrodes to use as the reference electrode during a monitoring session for the patient; and control the plurality of electrodes to detect the at least one of maternal or fetal biopotential signals using the selected one of the plurality of electrodes as the reference electrode in each of the plurality of electrode pairs.

In another example, the disclosure describes a method for non-invasively acquiring at least one of maternal or fetal biopotential signals comprising: receiving, by processing circuitry and during a monitoring session during which a wearable device including a plurality of electrodes is worn about the torso of a pregnant patient, the at least one of maternal or fetal biopotential signals detected by a plurality of electrode pairs selected from the plurality of electrodes of the wearable device, wherein a first electrode of each of the plurality of electrode pairs is a measurement electrode and a second electrode of each of the plurality of electrode pairs is a reference electrode; selecting, by the processing circuitry and based on processing the at least one of maternal or fetal biopotential signals detected by the plurality of electrode pairs, one of the plurality of electrodes to use as the reference electrode during the monitoring session; and controlling, by the processing circuitry, the plurality of electrodes to detect the at least one of maternal or fetal biopotential signals using the selected one of the plurality of electrodes as the reference electrode in each of the plurality of electrode pairs.

In another example, the disclosure describes a non-transitory computer readable medium comprising instructions that when executed by processing circuitry cause the processing circuitry to: receive, during a monitoring session during which a wearable device including a plurality of electrodes is worn about the torso of a pregnant patient, at least one of maternal or fetal biopotential signals detected by a plurality of electrode pairs selected from the plurality of electrodes of the wearable device, wherein a first electrode of each of the plurality of electrode pairs is a measurement electrode and a second electrode of each of the plurality of electrode pairs is a reference electrode; select, based on processing the at least one of maternal or fetal biopotential signals detected by the plurality of electrode pairs of the wearable device, one of the plurality of electrodes to use as the reference electrode during the monitoring session; and control the plurality of electrodes to detect the at least one of maternal or fetal biopotential signals using the selected one of the plurality of electrodes as the reference electrode in each of the plurality of electrode pairs.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a diagram of an example system for the acquisition and communication of at least one of maternal or fetal biopotential signals and prediction of at least one of maternal or fetal outcomes using trained machine learning model(s) in accordance with one or more techniques of the disclosure.

FIG. 2 is a more detailed block diagram of the electronic components of an example wearable device and an example patient computing device in accordance with one or more techniques of the disclosure.

FIG. 3 is a block diagram of an example fetal monitoring system (FMS) in accordance with one or more techniques of the disclosure.

FIG. 4 is a diagram of an example wearable device including a plurality of sensors (e.g., electrodes) embedded or affixed to a wearable garment or band in accordance with one or more techniques of the disclosure.

FIGS. 5A-5D are graphs illustrating an example mixed maternal-fetal ECG signal (FIG. 5A), a fetal ECG signal extracted from the mixed maternal-fetal ECG signal (FIG. 5B), a graph showing an identification of peaks in the fetal heart rate (FIG. 5C), and a presentation of fetal heart rate as determined based on the fetal ECG signal (FIG. 5D), respectively, in accordance with one or more techniques of the disclosure.

FIGS. 6A-6D are graphs showing an example abdominal signal obtained using the example wearable device (FIG. 6A), the envelope of the original uterine activity (UA) and smoothed UA signal (FIG. 6B), the detected uterine activity signal (FIG. 6C), and an example combined fetal heart rate and uterine signal graph (FIG. 6D), respectively, in accordance with one or more techniques of the disclosure.

FIG. 7 is a more detailed block diagram of the electronic components of an example wearable device, in accordance with one or more techniques of the disclosure.

FIG. 8 is a flow chart illustrating an example process by which a computing device may select a best electrode pair from which to obtain maternal/fetal ECG data for a patient, in accordance with one or more techniques of the disclosure.

### DETAILED DESCRIPTION

In general, the disclosure describes devices and methods for the non-invasive acquisition of at least one of maternal or fetal physiological signals associated with a pregnant patient and her fetus. The device may be part of one or more systems and/or methods for predicting maternal and/or fetal health outcomes based, at least in part, on the acquired maternal and/or fetal physiological data. The device may include a wearable device configured to be worn by a pregnant patient. The wearable device may include, for example, a wearable band configured to be worn about the torso of the patient.

The wearable device includes a plurality of electrodes configured to non-invasively sense maternal and/or fetal physiological signals, such as biopotential signals, associated with the patient and her fetus. The maternal and/or fetal physiological signals (also referred to herein as “patient data”) acquired by the wearable device may include, for example, data regarding sensed biopotential signals such as maternal and/or fetal electrocardiography (ECG) signals, maternal electromyography (EMG) signals, and/or other biopotential signals. The patient data acquired by the device may further include other sensed biometric data such as temperature, patient movement, breathing rate, blood pressure, skin impedance, glucose levels, pH blood levels, blood oxygen level, acoustic data (acquired using, for example, doppler ultrasound or other acoustic techniques), and any other sensed biometric parameter. The patient data considered by the overall system to predict maternal and/or fetal health outcomes may include, in addition to the patient data sensed by the wearable device, information regarding one or more maternal health conditions, including preexisting conditions such as gestational diabetes, high blood pressure, etc. In some examples, the patient data considered by the system may further include data obtained from a mental health assessment, a social determinates of health (SDoH) assessment, socio-economic data, etc. The patient data may further include any data that may be relevant for the prediction of maternal and/or fetal outcomes, and the disclosure is not limited in this respect.

Analysis of the maternal and/or fetal biopotential signals acquired by the wearable device and/or other patient data may assist clinicians in identification of features or patterns in patient data that could lead to sub-optimal outcomes, support real time decision-making by the clinical team, thus helping to promote timely, appropriate interventions, and decrease overall costs associated with adverse maternal and fetal outcomes. The techniques may aid clinicians and healthcare providers to improve prenatal care and to better manage risk of pregnancy patients while at home, allowing for continued monitoring and alert triggering. In addition, healthcare costs associated with pregnancy may be reduced by eliminating unnecessary travels and clinic visits, saving time and stress to future mothers. In addition, collection of relevant patient data may provide a framework for clinical and scientific research in the field of prenatal care and support continuous updates and refinements to the predictive models and the resulting predicted maternal and/or fetal outcomes.

In some examples, according to one or more techniques of the disclosure, a cloud-based pregnancy monitoring system receives the patient data acquired by the wearable device and/or other patient data, applies the patient data to one or more trained machine learning models trained to predict maternal and/or fetal health outcomes, and predicts one or more maternal and/or fetal outcomes associated with the pregnant patient based on the patient data. The machine learning model(s) are trained using a training data set including patient data and associated outcomes obtained for each of a plurality of patients (e.g., pregnant human mothers and their fetuses). The training data is indicative of features of the patient data that are predictive of one or more maternal or fetal outcomes (either adverse or non-adverse). The training data may be used to generate the one or more machine learning models for the identification of high-risk pregnancies, e.g., prediction of one or more adverse outcomes described herein.

The physiological data acquired by the wearable device according to the techniques of the disclosure, and/or other patient data, is input to the trained machine learning model (or models) to predict one or more maternal and/or fetal outcomes. Predicted fetal outcomes may include, but are not limited to, heart defects, fetal arrythmia, fetal cardiac anomalies or other fetal heart conditions, preterm labor, term labor, Apgar scores (e.g., 1, 5 and 10 minutes after birth), cord blood gas pH level, neonatal destination immediately after birth, admission to Neonatal Intensive Care Unit (NICU) within 48 hours of birth, NICU length of stay, resuscitation intervention, other neonatal complications, neonatal death up to 28 days after birth, etc. Predicted maternal outcomes may include, but are not limited to, peripartum cardiomyopathy and/or other maternal heart conditions, preterm labor, term labor, magnesium and/or potassium abnormalities (e.g., hyper or hypo), mode of delivery (e.g., vaginal or C-section), reason for C-section, grade of C-section (If performed—Grades 1, 2, 3 or 4), length of stay, destination immediately after birth, admission to a higher level of care, complications (type and severity), hour of day of delivery, day of week of delivery, etc. The trained machine learning models may further be trained to distinguish between Braxton-Hicks contractions (also referred to as false labor contractions) versus true labor contractions.

In some examples, one or more techniques of the disclosure combine patient data acquired by the wearable device, such as maternal and/or fetal biopotential signals (e.g., ECG, EMG, and/or heart rate data), biometric data such as uterine contraction data, blood pressure, weight, glucose, pH blood levels, blood oxygen level, breathing rate, patient movement, and/or temperature, patient health assessment data such as results of a mental health assessments, a social determinates of health (SDoH) assessment, data regarding preexisting conditions, patient usage patterns (for example, the timing or update patterns when answering questions on a psychological survey), time of day, frequency or time between measurements, and/or any other patient data relevant to prediction of maternal and/or fetal outcomes for use as training data and/or input data for a current monitoring session for which one or more outcomes are predicted.

The data acquired by the wearable device according to the techniques of the disclosure may help identify false predictions of fetal distress that may lead to unnecessary Cesarean sections, thus helping to minimize performance of unnecessary C-Sections and the associated increase in health care costs and maternal recovery time. At the same time, accuracy regarding the prediction of actual fetal distress may be maximized, allowing for timely interventions. The techniques of the disclosure thus provide a comprehensive and accurate monitoring system that takes many types, attributes, features, and/or patterns of maternal and/or fetal data into account when predicting one or more maternal and/or fetal outcomes.

In some examples, the wearable device may be used by a pregnant patient in a home or other non-clinical environment to perform one or more remote monitoring sessions for the acquisition of maternal and/or fetal biopotential (such as ECG and/or EMG) data, heart rate data, and/or other physiological data. This remote monitoring, in combination with a cloud-based remote monitoring system (e.g., telehealth and/or telemedicine system), may improve the mother's comfort and peace of mind during pregnancy. The wearable device may be used to monitor the health of prenatal and postpartum patients in a remote clinical or non-clinical setting. The wearable device may also be used during labor and delivery in addition to or instead of a traditional cardiotocography (CTG) monitoring device in clinical/hospital environment.

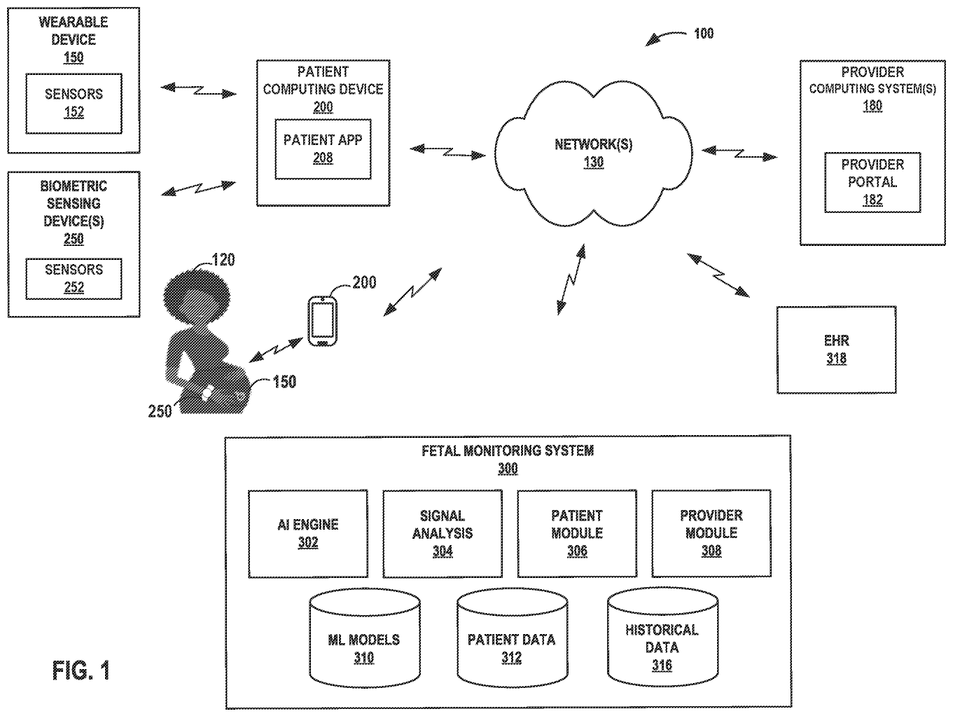

FIG. 1 is a diagram of an example system 100 for the acquisition and communication of patient data and/or prediction of maternal and/or fetal outcomes using trained machine learning model(s) in accordance with one or more techniques of the disclosure. In this example, system 100 includes a wearable device 150 including a plurality of sensors 152 (e.g., electrodes) configured to sense physiological signals of a patient 120 (a pregnant human mother and/or her fetus). The physiological signals may include, for example, maternal and/or fetal biopotential signals, such as ECG signals or other signals indicative of maternal and/or fetal cardiac activity, EMG signals indicative of uterine activity or contractions, or any other sensed physiological data. System 100 further includes at least one patient computing device 200, provider computing system 180, and a cloud-based fetal monitoring system (FMS) 300.

In some examples, wearable device 150 includes a wearable (e.g., a garment or a band) configured to be worn about the torso of a pregnant patient 120, one or more sensors 152 affixed or embedded in the wearable, a communications interface, and a controller. The one or more sensors 152 are configured to sense physiological signals, such as one or more biopotential signals of the mother and/or the fetus, such as ECG and/or EMG signals. In some examples, the sensed physiological data includes maternal and/or fetal ECG or heart rate data; however, the disclosure is not limited in this respect. Wearable device 150 is configured to wirelessly communicate sensor data representative of the sensed physiological signals for receipt by at least one computing device, such as patient computing device 200. The wearable device controller is configured to control signal acquisition from the one or more sensors and to control wireless communication of the sensor data.

Patient computing device 200 is configured for wireless communication with wearable device 150. For example, patient computing device 200 wirelessly receives the sensor data transmitted by wearable device 150. In some examples, patient computing device 200 may include one or more personal computing devices of the patient 120. For example, patient computing device 200 may include a mobile computing device (e.g., smartphone, tablet, or laptop computer), a desktop computer, a smartwatch, etc. Computing device 200 and wearable device 150 may communicate using, for example, the Bluetooth® or Bluetooth® Low Energy (BLE) protocols, near field communication (NFC), Wi-Fi, or any other form of wireless and/or wired communication.

In some examples, patient computing device 200 includes a patient application 208 stored in a memory or other data storage device of patient computing device 200 as a computer-readable medium comprising instructions that, when executed by patient computing device 200, generates one or more interactive pages for display on a user interface of patient computing device 200. The one or more interactive pages guide the patient through a monitoring session during which physiological signals are acquired by wearable device 150 and corresponding sensed patient data is communicated from wearable device 150 to patient computing device 200. Example systems and methods for remote pregnancy monitoring and management are shown and described in U.S. Patent Application No. 63/264,775, filed Dec. 1, 2022, which is incorporated by reference herein in its entirety.

Patient computing device 200 is further configured to communicate with a variety of other devices or systems via network(s) 130. For example, computing device 200 may be configured to communicate with one or more computing systems, e.g., provider computing system 180 and/or Fetal Monitoring System (FMS) 300.

FMS 300 includes an AI engine 302, a signal analysis module 304, a patient module 306, and a provider module 308. FMS 300 further includes or is associated with one or more databases or other storage device(s) that store one or more stored machine learning (ML) model(s) 310, patient data 312, sensor data 314, and historical data 316. Sensor data 314 includes the raw data representative of the biopotential signals detected by wearable device 150 during one or more patient monitoring sessions. Patient data 312 includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical data 316 includes historical maternal and/or fetal patient data associated with a plurality of patients. FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302 of FMS 300 is configured to train one or more machine learning (ML) model(s) 310 based on historical data 316 associated with a plurality of patients to generate one or more maternal and/or fetal outcome predictions. AI engine 302 is further configured to determine, based on processing patient data for a pregnant patient using one or more ML models 310 trained with the historical data 316, one or more maternal and/or fetal outcome predictions for the pregnant patient. Example systems and methods of training of the one or more machine learning models and or prediction of one or more maternal and/or fetal outcomes are described in U.S. patent application Ser. No. 17/457,206, filed Dec. 1, 2021, U.S. Provisional Patent Application 63/265,952, filed Dec. 23, 2021, and U.S. Provisional Patent Application 63/268,244, filed Feb. 18, 2022, each of which is incorporated by reference herein in its entirety.

Patient computing device(s) 200 may transmit data, including patient data received from wearable device 150, to computing system(s) 180 and/or FMS 300 via network(s) 130. The data may include sensed patient data, e.g., values of one or more biopotential signals, such as ECG and/or EMG signals, sensed by wearable device 150 and other physiological signals or data sensed or otherwise determined by wearable device 150 and/or patient computing device(s) 200. FMS 300 may retrieve data regarding patient(s) from one or more sources of electronic health records (EHR) 318 (which may also be referred to as electronic medical records, EMR) via network 130. EHR 318 may include data regarding historical (e.g., baseline) patient data, previous health events and treatments, preexisting conditions, disease states, comorbidities, demographics, height, weight, and body mass index (BMI), as examples, of patients. FMS 300 may use data from EHR 318 to configure algorithms implemented by wearable device 150, patient computing device 200 and/or FMS 300 to control acquisition of the sensed biopotential signals from wearable device 150 during a monitoring session and/or to predict maternal and/or fetal outcomes based on patient data acquired during a monitoring session for a patient.

Network(s) 130 may include, for example one or more local area networks (LANs), wireless local area networks (WLANs), virtual private networks (VPNs), wide area networks (WANs), the Internet, etc. Network(s) 130 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network(s) 130 may include one or more networks administered by service providers and may thus form part of a large-scale public network infrastructure, e.g., the Internet. Network(s) 130 may provide computing devices and systems, such as those illustrated in FIG. 1, access to the Internet, and may provide a communication framework that allows the computing devices and systems to communicate with one another. In some examples, network(s) 130 may include a private network that provides a communication framework that allows the computing devices and systems illustrated in FIG. 1 to communicate with each other but isolates some of the data flows from devices external to the private network for security purposes. In some examples, the communications between the computing devices and systems illustrated in FIG. 1 are encrypted.

Provider computing system 180 includes one or more computing devices used by providers (e.g., physicians, physician assistants, nurses, nurse midwives, pharmacists, therapists, clinical support staff, etc.) to view patient data gathered or generated during one or more patient monitoring sessions, including one or more maternal and/or fetal outcome predictions associated with the patient monitoring sessions, for one or more patients. For example, provider computing system 180 may include a provider portal 182 stored in a memory or other data storage device of provider computing system 180 as a computer-readable medium comprising instructions that when executed by provider computing system 180 generates one or more interactive pages for display on a user interface of provider computing system 180 that allow health care providers to view raw and/or processed patient data or other data generated by analysis of the patient data, including one or more predicted maternal and/or fetal outcomes, for one or more patients.

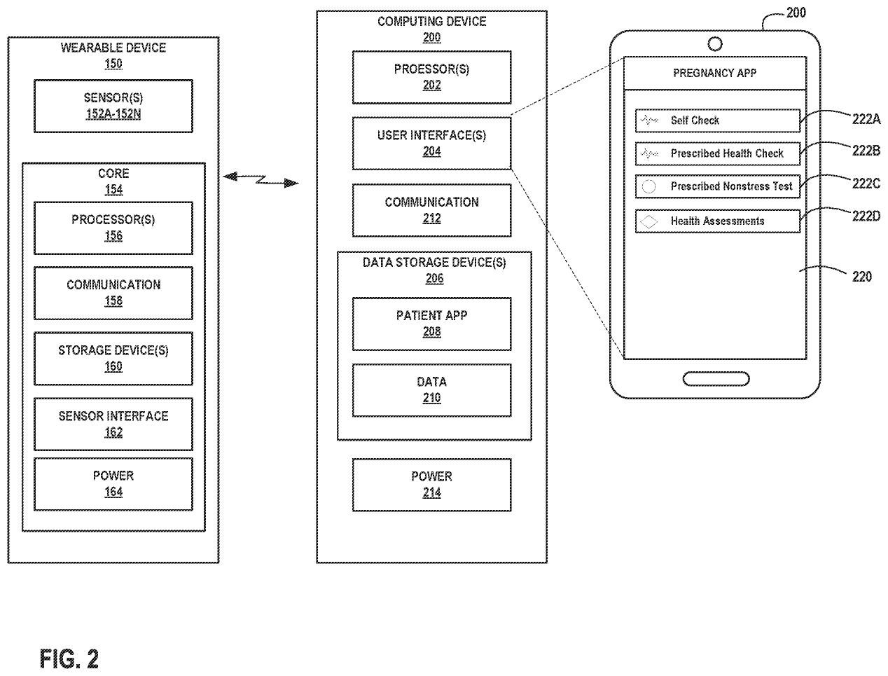

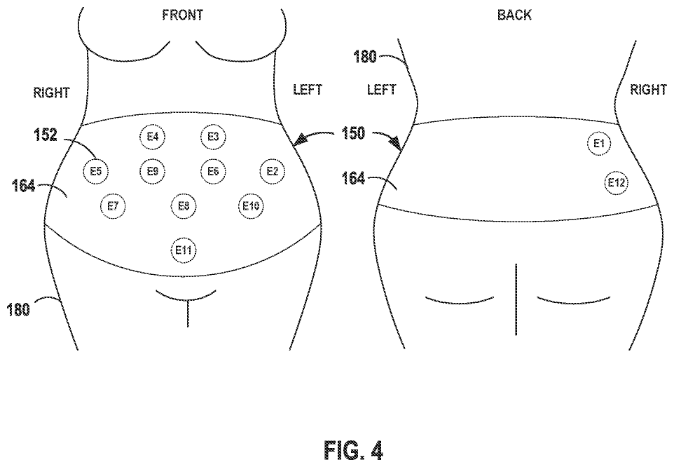

FIG. 2 is a more detailed block diagram of the electronic components of an example wearable device 150 and an example patient computing device 200 in accordance with one or more techniques of the disclosure. Wearable device 150 includes one or more sensors 152 (e.g., electrodes) configured to sense physiological signals of a patient, such as maternal and/or fetal biopotential signals, such as ECG and/or EMG signals. In some examples, wearable device 150 includes a wearable (e.g., a garment or band 164 such as shown in FIG. 4) including a plurality of electrodes or other sensing devices 152A-152N affixed to or embedded therein.

In some examples, the sensors are configured to sense at least one of maternal or fetal biopotential signals, such as at least one of maternal or fetal ECG signals. In other examples, one or more of the sensors may be configured to sense any one or more of cardiotocography (CTG) signals, electromyography (EMG) signals, EMG myometrium signals, pulse oximeter signals, respiratory inductance plethysmography (RIP) (thoracic and abdominal) signals, acoustic signals, actigraphy signals, temperature information (temperature sensor(s)), accelerometer or movement information, photoplethysmography (PPG) (e.g., optical measurement for pulse rate and SpO2), and/or any other biopotential or physiological signal or parameter of the patient. The sensors 152 may thus further include any appropriate sensor(s) configured to detect or sense any of the listed signals or physiological parameter associated with the patient.

Wearable device further includes control electronics that process the sensed physiological signals of the patient acquired by sensors 152 and communicate the sensed patient data for receipt by patient computing device 200. In some examples, the control electronics are packaged in a core 154 configured to be removably connected to the wearable garment or band. To that end, core 154 includes one or more processors 156, a communication interface 158, storage devices 160, a sensor interface 162, and a power source 164 (e.g., one or more batteries). Sensor interface 162 includes circuitry configured to receive sensor data corresponding to the physiological signals detected by the one or more sensors 152. Communication interface 158 is configured to support wireless communication between wearable device 150 and one or more computing devices, such as patient computing device 200. Storage devices 160 include one or more hardware memories or other data storage devices configured to store executable control instruction and/or raw sensor data associated with one or more monitoring sessions. Wearable device 150 may store sensor data temporarily during each monitoring session for wireless transmission to a computing device, or wearable device may store sensor data associated with multiple monitoring sessions for later transmission to a computing device.

Patient computing device 200 includes one or more processor(s) 202, a user interface 204, communication interface 212, data storage devices 206, and a power source 214 (e.g., one or more batteries). In some examples, patient computing device 200 may include one or more personal computing devices of the patient. For example, patient computing device 200 may include a mobile computing device (e.g., smartphone, tablet, or laptop computer), a desktop computer, a smartwatch, etc. Communication interface 212 of patient computing device 200 is configured for wireless communication with wearable device 150. For example, communication interface 212 and communication interface 158 of wearable device 150 may be configured to communicate using, for example, the Bluetooth® or Bluetooth® Low Energy (BLE) protocols, near field communication (NFC), or any other form of wireless communication.

Patient computing device 200 includes a patient application 208 stored in data storage device(s) 206. For example, patient application 208 may include a computer-readable medium comprising instructions that, when executed by one or more processor(s) 202 of patient computing device 200, generates one or more interactive pages for display on a user interface 204 of patient computing device 200 that guide the patient through a monitoring session during which physiological signals are acquired by wearable device 150 and corresponding sensor data is communicated from wearable device 150 to patient computing device 200. As shown in the example of FIG. 2, example patient computing device 200 includes a touch screen display 220 on which one or more interactive pages of a guided patient monitoring session are displayed. Each interactive page may include one or more user interface elements, such as user interface elements 222A-222D, by which a user may interact with patient application 208 (and thus with wearable device 150 and/or FMS 300) to conduct one or more monitoring sessions including a self-check, a prescribed health check, a prescribed nonstress test, and/or one or more health assessments, such as one or more mental health assessments, social determinants of health assessments, socio-economic assessments, etc. Example systems and methods for remote pregnancy monitoring and management are shown and described in U.S. Patent Application No. 63/264,775, filed Dec. 1, 2022, which is incorporated by reference herein in its entirety.

Communication interface 204 of patient computing device 200 is further configured to communicate with a variety of other devices or systems via network(s) 130 (see FIG. 1). For example, computing device 200 may be configured to communicate with one or more computing systems, e.g., one or more of provider computing system 180 and/or FMS 300.

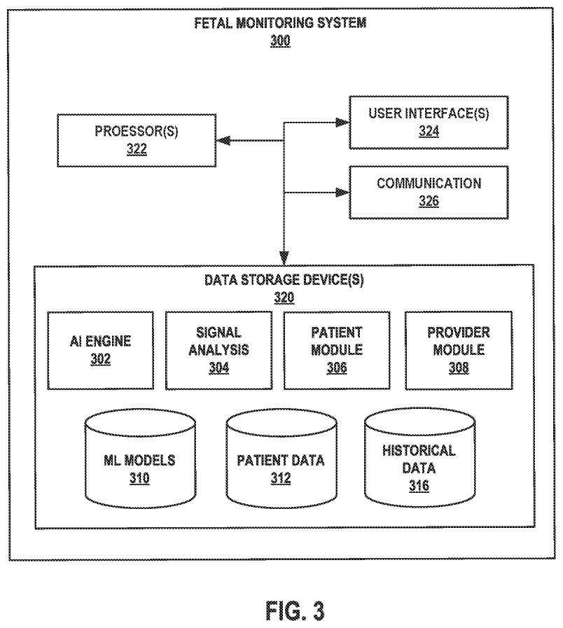

FIG. 3 is a block diagram of an example fetal monitoring system (FMS) 300 in accordance with one or more techniques of the disclosure. FMS 300 includes one or more processors 322, user interfaces 324 by which one or more users may interact with FMS 300, communication interfaces 326 which provide for communication with one or more computing devices such as patient computing device 200 and/or provider computing systems 180, and one or more data storage devices 320. Data storage devices 320 include storage for one or more computing modules including AI engine 302, signal analysis module 304, patient module 306, and provider module 308. FMS 300 further includes or is associated with one or more databases or other storage device(s) that store one or more stored machine learning (ML) model(s) 310, patient data 312, and historical data 316. Patient data 312 includes, for each of a plurality of patients, biopotential or other physiological patient data sensed by wearable device 150 during one or more patient monitoring sessions, biometric data associated with the patient, and/or patient data obtained during one or more health assessment sessions. Patient data 312 also includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical data 316 includes historical patient data associated with a plurality of patients. Processor(s) 322 of FMS 300 execute provider module 308 to provide remote provider-facing fetal monitoring services that support healthcare provider interaction with FMS 300 via provider portal 182 of provider computing system(s) 180. Similarly, processors 322 of FMS 300 execute patient module 306 to provide remote patient-facing fetal monitoring services that support patient interaction with FMS 300 via patient application 208 of patient computing device 200.

Signal analysis module 304 may apply one or more signal processing or preprocessing techniques to the raw sensor data representative of the maternal and/or fetal biopotential signals acquired by the one or more sensors. For example, signal analysis module 304 may apply normalization, denoising, filtering, artifact detection and/or artifact correction to any one or more of the sensed signal data received from the wearable device 150. Signal analysis modules may also perform feature extraction for the sensed biopotential signals including for example, extraction of a fetal ECG signal from a mixed maternal-fetal ECG signal, identification of one or more features of the maternal and/or fetal ECG signals including, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). Signal analysis module 304 may further extract one or more features of the maternal and/or fetal heart rate signals including but not limited to, for example, baseline heart rate, baseline variability, fetal heart rate variability, number of accelerations per second, number of early, late, and variable decelerations per second, number of prolonged decelerations per second, sinusoidal pattern, etc.

FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302, when executed by processors 322 of FMS 300, is configured to train one or more machine learning (ML) model(s) 310 based on historical data 316 associated with a plurality of patients to generate one or more maternal and/or fetal outcome predictions. AI engine 302, when executed by processors 322, is further configured to determine, based on processing patient data for a pregnant patient using one or more ML models 310 trained with the historical data 316 corresponding to a plurality of patients, one or more maternal and/or fetal outcome predictions for the pregnant patient.

Although in the examples described herein FMS 300 is described as performing the training of the ML models 310 and/or application of the models 310 to predict one or more maternal or fetal outcomes, some or all of the functions described herein as being performed by FMS 300 may be performed by any one or more of wearable device 150, patient computing device 200, provider computing system 180, or any other remote, local or distributed computing device or system, and that the disclosure is not limited in this respect. In addition, the various functions performed by FMS 300 may be implemented using a single computing device or system or they may be distributed across multiple computing devices or systems.

FIG. 4 is a diagram of an example wearable device 150 including a plurality of sensors 152A-152N (labeled E1-E12 and referred to generally as sensors 152) embedded or affixed to a wearable garment 164 in accordance with one or more techniques of the disclosure. In this example, the sensor configuration includes a total of twelve electrodes E1-E12 affixed to or embedded within garment 164 such that, when properly worn about the torso of a patient 120, the electrodes 152 are positioned about the torso of patient 120. One or more of the electrodes 152 may be positioned on the front, left side, right side and/or back of the patient 120. As shown in FIG. 4, in some examples, the sensor configuration on garment 164 is such that two electrodes are positioned on the back of patient 120 (E1 and E12 in this example). The number and configuration of sensors 152 on the wearable garment 164 may vary from that shown in FIG. 4, and the disclosure is not limited in this respect.

In order to capture maternal and fetal biopotential signals of sufficient quality, sensors 152 should provide good contact with the patient's skin, minimize sensor movement relative to the skin, and reduce signal noise from light movements of the patient. In some examples, one or more of sensors 152 include SilverBumps® dry electrodes available from Orbital Research, Inc. Example wearable garments that may be used to implement wearable device 150 are described in U.S. Pat. No. 9,579,055, issued Feb. 28, 2017, which is incorporated by reference herein in its entirety.

In other examples, instead of or in addition to dry electrodes, wearable device 150 may include any other type of sensing material or device to acquire the biopotential signals data, such as one or more of nanotechnology sensing devices, textile or silicon-based dry electrodes, nanotube sensors, cardiotocography (CTG) doppler transducers for acquiring signals associated with uterine contractions, and/or any other sensor that may be used to capture maternal and/or fetal biopotential signals.

In accordance with one or more techniques of the disclosure, the physiological (e.g., biopotential) signals sensed by wearable device 150 and analyzed to determine the status of the fetus and/or predict one or more maternal and/or fetal outcomes may include, but are not limited to, fetal heart rate (fHR), maternal heart rate (mHR), fetal ECG, maternal ECG, and maternal EMG signals.

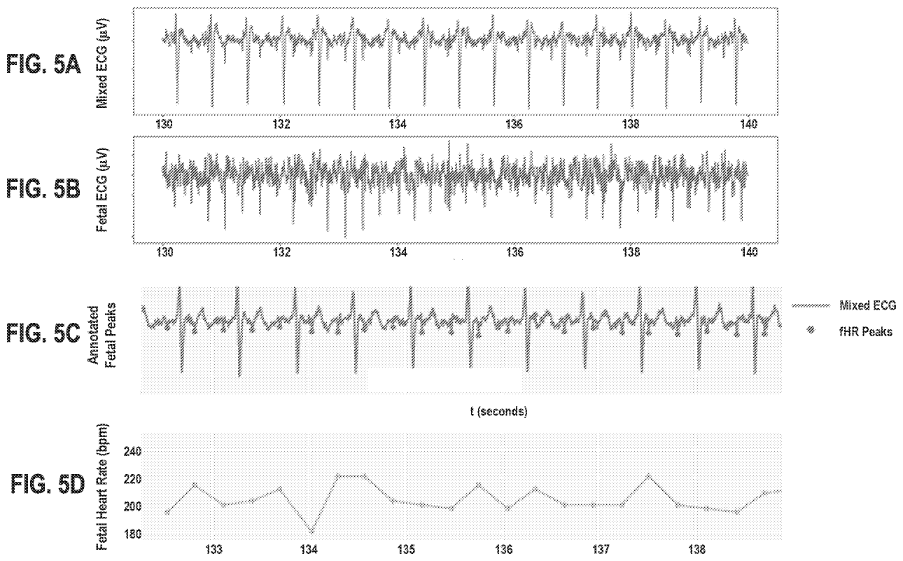

FIGS. 5A-5D are graphs illustrating an example mixed maternal-fetal ECG signal (FIG. 5A), a fetal ECG signal extracted from the mixed maternal-fetal ECG signal (FIG. 5B), a graph showing an identification of peaks in the fetal heart rate (FIG. 5C), and a presentation of fetal heart rate as determined based on the fetal ECG signal (FIG. 5D), respectively, in accordance with one or more techniques of the disclosure.

To obtain the fetal ECG (FIG. 5B) from the mixed (maternal and fetal) ECG signal (FIG. 5A) that is captured by the wearable device, an extraction algorithm may be employed. Example techniques for extracting a fetal ECG signal (FIG. 5B) from a mixed maternal-fetal ECG signal are described in U.S. Pat. No. 11,224,375, issued Jan. 18, 2022, which is incorporated by reference herein in its entirety.

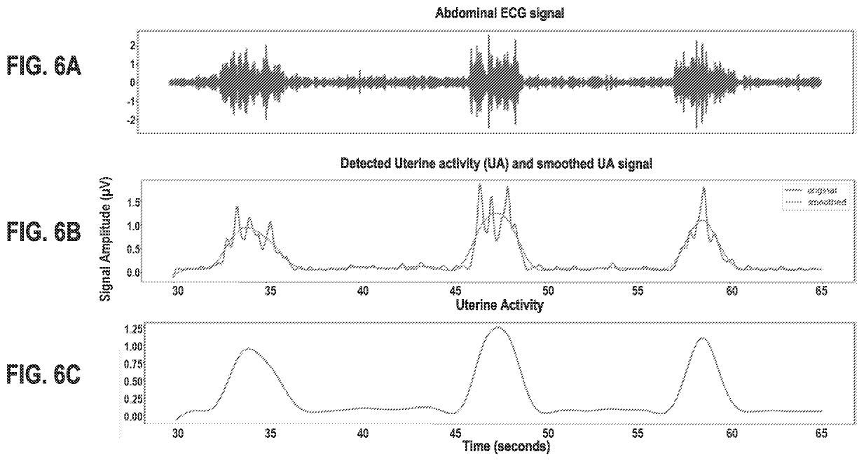

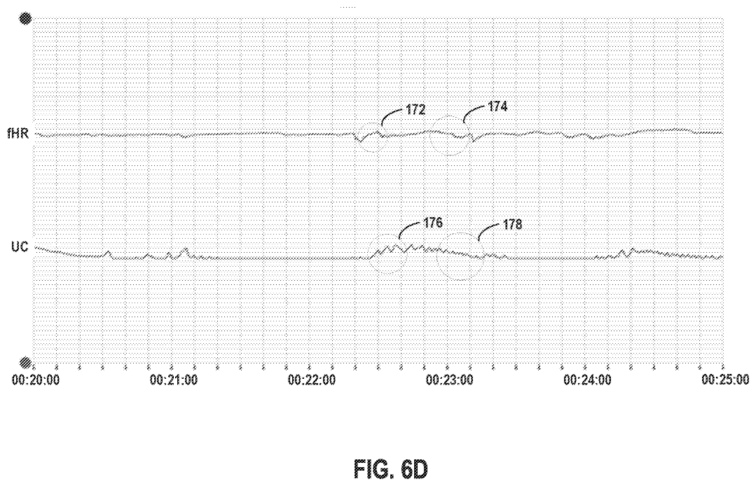

FIGS. 6A-6C are graphs showing an example abdominal (e.g., EMG) signal obtained using the example wearable device 150 as shown in FIG. 4 (FIG. 6A), the envelope of the original uterine activity (UA) and smoothed UA signal (FIG. 6B) and the detected uterine activity signal (FIG. 6C), in accordance with one or more techniques of the disclosure.

FIG. 6D is a combined fetal heart rate and uterine signal graph in accordance with one or more techniques of the disclosure. Example features of the fetal heart rate signal (upper portion of the graph) are indicated by reference numerals 172 and 174 and example features of the uterine contraction signal (lower portion of the graph) are identified by reference numerals 176 and 178. Reference numeral 172 indicates an acceleration of the fetal heart rate signal that occurred during a first period of time. Reference numeral 174 indicates a deceleration of the fetal heart rate signal that occurred during a second period of time. The detected accelerations/decelerations of the fetal heart rate signal (or the average, mean or other statistical characterization of the detected accelerations/decelerations) may be extracted as a feature that is input to the ML models for the prediction of maternal and/or fetal outcomes. Reference numeral 176 indicates the start of a uterine contraction substantially corresponding to the first period of time during which the fetal heart acceleration indicated by reference numeral 172 occurred. Reference numeral 178 indicates the end of the uterine contraction substantially corresponding to the second period of time during which the fetal heart rate deceleration 174 occurred.

The system may extract one or more features of the sensed biopotential signals and use the extracted features as inputs to a machine learning model (such as ML model(s) 310) to predict one or more maternal and/or fetal outcomes. For example, features of the fetal heart rate may include, but are not limited to, baseline heart rate, baseline variability, number of accelerations per second, number of early, late, and variable decelerations per second, number of prolonged decelerations per second, sinusoidal pattern, etc. Features of the fetal ECG may include, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). In another example, analysis of the raw fetal ECG signal may be considered as well to avoid the information loss associated with such feature extraction procedures.

Similar features may also be identified for the maternal heart rate. Uterine contraction (UC) features may include baseline uterine tone, contraction frequency, start/end time of uterine contractions, amplitude of uterine contractions, duration of uterine contractions, and strength (intensity) of uterine contractions.

Example features of the fetal heart rate may include, but are not limited to, the features shown in Table 1. Similar features may also be identified with respect to the maternal heart rate.

TABLE 1
Variable Description (fHR)

Fetal heart rate baseline (beats per minute)
Number of accelerations
Number of fetal movements
Number of uterine contractions
Number of moderate decelerations
Number of severe decelerations
Number of prolonged decelerations
Percentage of time with abnormal short-term variability
Mean duration of short-term variability
Mean duration of long-term variability
Percentage of time with abnormal long-term variability
Histogram tendency
Fetal state class code (N = Normal, S = Suspected, P = Pathological)
Width of FHR histogram
Minimum of FHR histogram
Maximum of FHR histogram
Number of histogram peaks
Number of histogram zeroes
Histogram mode
Histogram median
Histogram variance
Amplitude of FHR

The patient data for a particular patient may include patient data obtained during one or more previous monitoring sessions for the patient. The patient data associated with the previous monitoring sessions may thus be used to establish one or more baselines for the patient. For example, baselines with respect to maternal ECG and/or heart rate, fetal ECG and/or heart rate, etc., may be established and used as feature inputs to one or more ML models for prediction of maternal and/or fetal outcomes for the patient. In this way, longitudinal information for the patient over time may be taken into account when determining the one or more maternal and/or fetal outcome predictions for the patient.

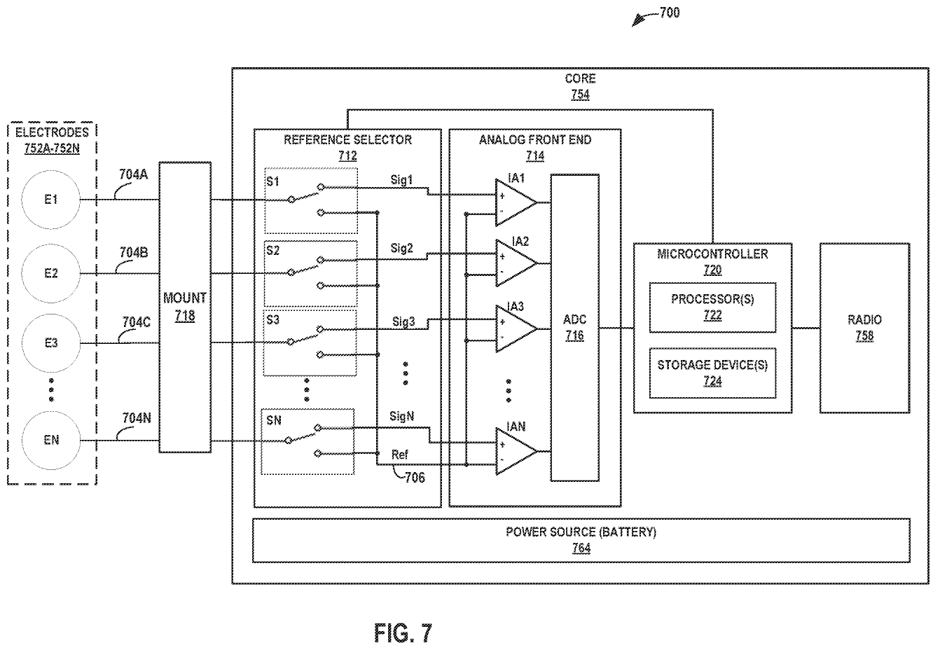

FIG. 7 is a more detailed block diagram of the electronic components of an example wearable device 700, in accordance with one or more techniques of the disclosure. In some examples, wearable device 700 may be used to implement wearable device 150 of FIGS. 1, 2, and 4. Example wearable device 700 includes one or more electrodes 752A-752N (E1-EN), a mount 718, and a core 754 including the control electronics for wearable device 700. In this example, core 754 is configured to be removably connected to a wearable garment or band (such as shown in FIG. 4) via a mount 718. However, in some examples, the core need not necessarily be configured to be removably connected to the wearable garment or band, and the disclosure is not limited in this respect.

In this example, core 754 includes reference selector circuitry 712, analog front end circuitry (AFE) 714, a communication interface or radio 758, a power source 764 (e.g., one or more batteries), and a computational unit (e.g., a microcontroller) 720 including one or more processor(s) 722 and one or more storage device(s) 724. Storage device(s) 724 are configured to store executable control instructions and/or raw sensor data associated with one or more monitoring sessions. In some examples, wearable device 700 may store sensor data temporarily during each monitoring session for wireless transmission to a computing device, or wearable device 700 may store sensor data associated with multiple monitoring sessions for later transmission to a computing device.

The plurality of electrodes (E1-EN) 752A-752N are connected via wire connectors 704A-704N, respectively, to mount 718, which interfaces to the core 754, containing the measurement electronics. In the example implementation of FIG. 7, each electrode of the plurality of electrodes 752A-752N is wired to a series of “N” digitally controlled switches S1-SN included in reference selector circuitry 712. In some instances, these switches S1-SN may be integrated into a single chipset with AFE 714. In other instances, they may be independent of AFE 714.

In some examples, the switches S1-SN are single-pole dual throw, where one throw (labeled Sig1-SigN) is connected to the positive input of a corresponding (1 of N) instrumentation amplifier (labeled IA1-IAN) and the other throw is tied to a signal path labeled “Ref,” which is tied to the negative input of all the instrumentation amplifiers. Each instrumentation amplifier then measures the difference between the positive input and the reference signal. Other example implementations may include multi-pole, multi throw switches to create more complex configurations.

In some examples, a single electrode of the total N is selected as the reference electrode. In other instances, a plurality (2 or more) electrodes may be combined to form a reference “plane” against which the other electrodes are measured.

In further examples, electrodes may be paired on an instrumentation amplifier such that one electrode is the positive input, and one electrode is the negative or “reference” input. In such examples the “negative” electrode is not reused (e.g. Electrode E1 and E2 feed IA1, Electrode E3 and E4 feed IA2, etc.). This example may be further expanded such that the “Ref” electrode feeds the negative input to some, but not all, instrumentation amplifiers (e.g., Electrode E1 is the + of IA1, Electrode E2 is the + of IA2, and Electrode E3 is the − of IA1 and IA2; Electrode E4 is the + of IA3, and Electrode E5 is the + of IA4, while Electrode E6 is the − of IA3 and IA4). This approach can be expanded cover any combination of N−1:1 electrodes as Signal:Reference through 1:N−1 electrodes as Signal:Reference. Although several example implementations of wearable device 700 are described herein, the disclosure is not limited in this respect.

After the digitally controlled switches S1-SN are set and signals are routed through the instrumentation amplifiers IA1-IAN, the measurement results are then read through an analog to digital converter (ADC), which may be integrated into AFE 714. The digital versions of the measurements are then processed via microcontroller 720 and then passed on for wireless communication via the radio 758.

In addition to the N−1 physical channels created, all unique combinations of N−1 electrodes can be created mathematically leading to an additional set of “virtual” channels to be monitored.

The reference selector switches S1-SN are programmed from microcontroller 720. The various selection schemes discussed above may be decided by several different methods including but not limited to, hard selection, one-time algorithms, sliding algorithms, and machine learning.

Hard selection is a choice made prior to firmware build to select a single electrode to always be the reference. In this case the reference electrode is decided prior to firmware build and is not changed unless a new firmware update is provided in which a new microcontroller image is provided to change the reference electrode.

One-time algorithms refer to an instance in which data is collected during a predetermined period of time during which several combinations of reference electrodes are used. This period of time may be at or near the beginning of a monitoring session and may be referred to as an initialization period of the monitoring session. In some examples, a parameter-based decision tree is then used to select the final reference electrode depending on the parameter being optimized. Parameters may include but are not limited to, a signal to-noise ratio (SNR) for the biopotential signal of interest, a noise-to-noise ratio of high frequency to low frequency noise, a baseline wander minimization, a lead-off elimination, and/or any combination of the above. These algorithms may be executed either by microcontroller 720 on board core 754, or in the cloud (e.g., by FMS 300 or other cloud-based computing device). In these examples, once the final configuration is determined it is used for the duration of a monitoring session.

Sliding algorithms refer to an instance where a microcontroller 720 (and/or FMS 300 or other cloud-based computing device) data stream is continually monitored for the optimization parameter (or parameters) of interest. The reference electrode (or electrodes) may then be changed in real time based on the identification of a more likely optimal configuration. The same parameters discussed above can be used as optimization parameters. The sliding algorithm continues to monitor and adjust the reference electrode (when determined necessary) throughout the duration of the monitoring session.

Machine learning (ML) algorithms refer to an instance where, instead of mathematical algorithms, ML models are used to predict an optimal reference electrode(s) combination. In some examples, simplified versions of these can be deployed internal to core 754 using ML focused chipsets, meanwhile more complex versions can be deployed in the cloud, where adequate processing power can be deployed. Further, the ML algorithms can be deployed in a one-time algorithm instance or sliding algorithm instance. Due to the nature of ML, many different combinations of parameters and observational variables can be used for optimization including, but not limited to, predicted accuracy of fetal heart rate, predicted accuracy of maternal heart rate, predicted yield of fetal heart rate, uterine activity directional differentiation, etc., and the disclosure is not limited in this respect.

During an initialization period, the computing device obtains maternal and/or fetal biopotential signal data for a patient from each of a plurality of electrode pairs selected from among the plurality of electrodes 752A-752N of the wearable device. In some examples, the total number of electrode pairs is given by the following equation:

$$Total Number of Possible Electrode Pairs = \frac{N !}{2 ( N - 2 ) !} = \frac{N ( N - 1 )}{2}$$

As one example, if N=11 (e.g., the wearable device includes 11 electrodes which may be used to measure the maternal/fetal biopotential signal(s), such as E1-E11 as shown in FIG. 4) there are 55 different reference-measurement electrode pair combinations. If the computing device determines that electrode E2 should be selected as the reference electrode, the following electrode pairs are used to acquire the maternal and/or fetal biopotential signals during the monitoring session:

### Pair 1: E1 to E2

### Pair 2: E3 to E2

### Pair 3: E4 to E2

### Pair 4: E5 to E2

### Pair 5: E6 to E2

### Pair 6: E7 to E2

### Pair 7: E8 to E2

### Pair 8: E9 to E2

### Pair 9: E10 to E2

### Pair 10: E11 to E2

In accordance with one or more techniques of the disclosure, in some examples, wearable device 700 may further implement a noise reduction approach for mixed-signal monitoring by using one of the electrodes 752A-752N as a driven electrode. The driven electrode is configured to supply a biasing voltage to a body of the patient. The biasing voltage is a low current, low voltage potential to bias the body to the same potential and reduce the impact of bias offset at each electrode measuring location.

For example, one of electrodes 752A-752N may be selected as the driven electrode. During a monitoring session, the driven electrode is supplied with a low current, low voltage potential signal to bias the body to the same potential and reduce the impact of bias offset at each electrode measuring location. In this way, each of the remaining electrodes of the plurality of electrodes 752A-752N are biased to the same potential regardless of their location on the body of the patient. This may help to cancel out common mode interference in the sensed maternal and/or fetal biopotential signal sensed by the remaining electrodes, resulting in a higher quality biopotential (e.g., ECG, EMG, etc.) signal and more accurate determination of fetal heart rate and generation of one or more predicted outcomes.

In some examples, the driven electrode may be adaptively selectable in a manner similar to that described above with respect to the reference electrode. For example, a computing device (such as any of FMS 300, wearable device 150/700, patient computing device 200, and/or provider computing system 180) may be configured to select, based on processing the maternal and/or fetal biopotential signal data sensed by each of the plurality of electrode pairs, one of the plurality of electrodes to use as the driven electrode during the monitoring session for the patient.

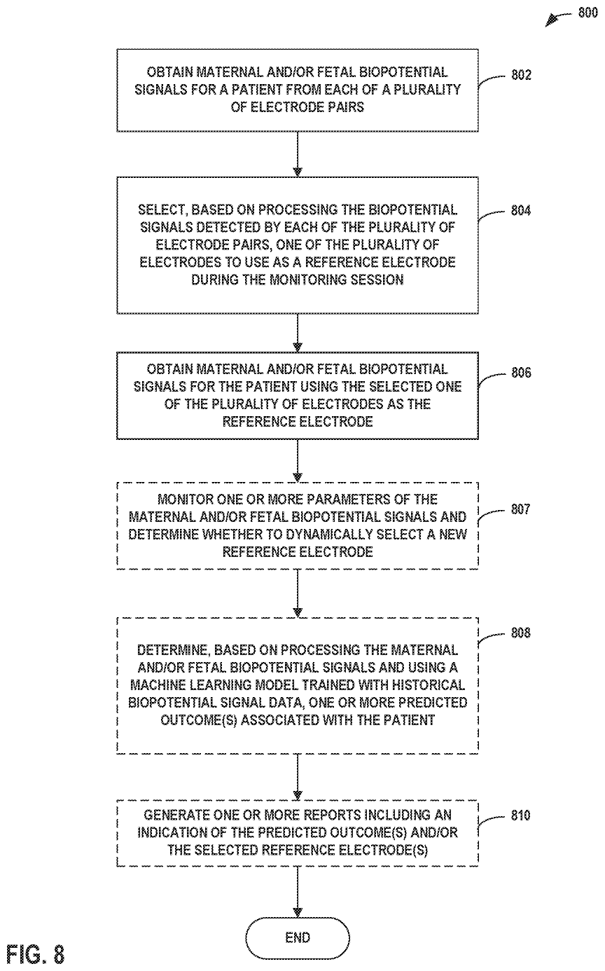

FIG. 8 is a flow chart illustrating an example process (800) by which a computing device, such as one or more processor(s) 302 of FMS 300, may select a reference electrode to use during a monitoring session to obtain maternal/fetal biopotential signal data for a patient, in accordance with one or more techniques of the disclosure. The computing device obtains maternal and/or fetal biopotential signal data for a patient from each of a plurality of electrode pairs (802). For example, the maternal and/or fetal biopotential signal data may be obtained from a wearable device, such as wearable device 150/700 including a plurality of sensors (e.g., electrodes) 152A-152N/752A-752N as shown and described herein. In some examples, the biopotential signal data may include data collected during an initialization period of a monitoring session. As described above with respect to the example wearable device of FIGS. 4 and 7, one electrode of each electrode pair may be referred to as a reference electrode and the other electrode of each electrode pair may be referred to as a measurement electrode. According to one or more techniques of the disclosure, the reference and measurement electrodes are switchable between two or more of the plurality of electrodes. For example, in a wearable device having 11 electrodes available for biopotential signal measurement, there will be (11×10)/2=55 possible electrode pairs, wherein one electrode in each electrode pair is the reference electrode and the other electrode in each electrode pair is the measurement electrode.

The computing device selects, based on processing the maternal and/or fetal biopotential signal data for the patient from each of the plurality of electrode pairs, one of the plurality of electrodes to use as a reference electrode during the monitoring session (804). This step further includes controlling the plurality of electrodes to detect the maternal and/or fetal biopotential signals using the selected one of the plurality of electrodes as the reference electrode in each of the plurality of electrode pairs. For example, the computing device may select the reference electrode based on one or more criteria described above, including a signal to noise ratio (SNR) of the received biopotential signals, or by any other method of selecting the reference electrode, resulting in high biopotential signal quality (or the best available biopotential signal quality). In some examples, the computing device selects the reference electrode based on processing of the maternal/fetal biopotential signal data from each electrode pair using a trained machine learning model trained using historical maternal/fetal biopotential signal data and information regarding features indicative of a quality maternal and/or fetal biopotential signal.

In some examples, the computing device continuously monitors, during the monitoring session, the maternal and/or fetal biopotential signal obtained from the plurality of electrode pairs, wherein each electrode pair includes the selected reference electrode. In some examples, the computing device dynamically changes the reference electrode during the monitoring session in response to detection of poor quality biopotential signals, or in response to detection that a different reference electrode is likely to provide higher quality biopotential signals. In such examples, the computing device further controls the plurality of electrodes to detect the maternal and/or fetal biopotential signals using the selected different one of the plurality of electrodes as the reference electrode in each of the plurality of electrode pairs.

In some examples, the computing device that analyzes the maternal/fetal biopotential signals received from the plurality of electrode pairs and selects a reference electrode from among the plurality of electrodes to be used during the monitoring session is a remote computing device, such as FMS 300. In other examples, one or both of wearable device 150/700 and/or a patient computing device, such as patient computing device 200, analyze the maternal/fetal biopotential signals received from the plurality of electrode pairs and selects the reference electrode pair to be used during a measurement period of the monitoring session.

During the monitoring session, the computing device obtains maternal and/or fetal biopotential signal data for the patient using the plurality of electrode pairs including the selected reference electrode and each of the remaining electrodes (806). In some examples, throughout the monitoring session, the computing device monitors one or more parameters of the maternal and/or fetal biopotential signals detected by the plurality of electrode pairs and based on the one or more parameters, determines whether to dynamically select a different one of the plurality of electrodes to use as the reference electrode (807). Based on the maternal and/or fetal biopotential signal data obtained for the patient during the monitoring session, the computing device determines, using a machine learning model trained with historical maternal and/or fetal biopotential signal data for a plurality of patients, one or more predicted maternal and/or fetal outcomes associated with the patient (808). The computing device may generate one or more reports including an indication of the predicted outcomes and/or the selected reference electrode(s) for display on one or more computing devices (810). For example, the computing device may generate the one or more reports for display on a user interface of a patient computing device or one a user interface of a provider computing system.

The process (800) may be repeated each time another monitoring session is performed for the patient.

The ability of the wearable device 150/700 to automatically and/or dynamically configure selection of the reference electrode provides one or more technical advantages. For example, the ability of the wearable device to configure selection of the reference electrode allows the wearable device to select the reference electrode that provides, in combination with each of the remaining electrodes, the best available maternal and/or fetal biopotential or other physiological signals. In addition, having the ability to adaptively change which electrode is selected as the reference electrode allows the system to dynamically respond to changing conditions (e.g., the wearable device and/or electrodes shift position on the patient, changes to the current and/or voltage potential of the patient's body, movement of the patient, etc.). In addition, the ability of the wearable device to change the selection of the reference electrode allows for the wearable device to avoid situations where certain electrodes may not be preferable selections as the reference. The wearable device may select an appropriate reference electrode based on one or more factors (e.g., best signal strength of fetal and/or maternal heartbeat, lowest signal to noise ratio, among other factors).

In addition, utilization of a driven electrode in a wearable device further provides one or more technical advantages. The usage of a driven electrode to provide a signal to bias the body may help reduce the noise in data collected by the electrodes of the wearable device by cancelling out common mode interference in the sensed maternal and/or fetal biopotential or other physiological signals. In some examples, the current and/or voltage potential of the human body may experience substantial swings, which can reduce the accuracy of devices that collect electrical data regarding the body. The usage of a driven electrode may thus result in higher quality maternal and/or fetal or other physiological signals for ongoing monitoring or analysis purposes.

Additional examples of components, devices, apparatus, methods, and/or systems which may be used in connection with one or more aspects of this disclosure are described in U.S. patent application Ser. No. 17/457,206, filed Dec. 1, 2021, U.S. Pat. No. 9,579,055, issued Feb. 28, 2017, U.S. Pat. No. 10,292,652, issued May 21, 2019, and U.S. Pat. No. 11,224,375, issued Jan. 18, 2022, each of which is incorporated herein by reference in its entirety.

In one or more examples, the functions described may be implemented in any combination of processing circuitry, including hardware, software, firmware, non-transitory computer-readable medium, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM or other optical disk storage, magnetic disk storage, or other magnetic storage devices, flash memory, or any other medium that can be used to store program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors and/or microcontrollers, ASICs, FPGAs, or other equivalent integrated or discrete logic circuitry, as well as any combination of such components. Accordingly, the term “processor,” as used herein may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware, and/or any other type or combination of processing circuitry.

Various examples have been described. These and other examples are within the scope of the following claims.

1. A system for non-invasively acquiring at least one of maternal or fetal biopotential signals comprising:
a wearable device configured to be worn by a pregnant patient, wherein the wearable device comprises a plurality of electrodes configured to detect at least one of maternal or fetal biopotential signals associated with the patient and her fetus; and
a computing device configured to:
receive at least one of maternal or fetal biopotential signal data detected by a plurality of electrode pairs of the wearable device, wherein each electrode pair includes two electrodes of the plurality of electrodes of the wearable device, and wherein a first electrode of each of electrode pair is a measurement electrode and a second electrode of each electrode pair is a reference electrode;
select, based on processing the at least one of maternal or fetal biopotential signal data detected by the plurality of electrode pairs of the wearable device, one of the plurality of electrodes to use as the reference electrode during a monitoring session for the patient; and
control the plurality of electrodes to detect the at least one of maternal or fetal biopotential signals using the selected one of the plurality of electrodes as the reference electrode in each of the plurality of electrode pairs.

2. The system of claim 1, wherein the wearable device further comprises a wearable band configured to be worn about a torso of the patient, wherein the plurality of electrodes are affixed to or embedded in the wearable band.

3. The system of claim 1, wherein the wearable device further comprises a driven electrode configured to supply a biasing voltage to a body of the patient.

4. The system of claim 3, wherein the computing device is further configured to:
select, based on processing the at least one of maternal or fetal biopotential signal data detected by one or more of the plurality of electrode pairs, one of the plurality of electrodes to use as the driven electrode during the monitoring session.

5. The system of claim 1, wherein the computing device selects one of the plurality of electrodes to use as the reference electrode during the monitoring session for the patient based on the at least one of maternal or fetal biopotential signal data detected by the plurality of electrode pairs during an initialization period of the monitoring session.

6. The system of claim 1, wherein the computing device controls the plurality of electrodes on the wearable device to detect the at least one of maternal or fetal biopotential signals using the selected one of the plurality of electrodes as the reference electrode during a measurement period of the monitoring session.

7. The system of claim 1, wherein the wearable device further includes at least one of a temperature sensor or an accelerometer.

8. The system of claim 1, wherein at one or more of the plurality of electrodes comprises a dry electrode.

9. The system of claim 1, wherein the computing device is further configured to automatically select, during the monitoring session and based on one or more parameters of the detected at least one of maternal or fetal biopotential signals, a different one of the plurality of electrodes to use as the reference electrode during the monitoring session.

10. The system of claim 9, wherein the computing device is further configured to control the plurality of electrodes to detect the at least one of maternal or fetal biopotential signals using the selected different one of the plurality of electrodes as the reference electrode in each of the plurality of electrode pairs.

11. The system of claim 1, wherein the at least one of maternal or fetal biopotential signals include one or more of maternal electrocardiography signals, fetal electrocardiography signals, or maternal electromyography signals.

12. A method for non-invasively acquiring at least one of maternal or fetal biopotential signals comprising:
receiving, by processing circuitry and during a monitoring session during which a wearable device including a plurality of electrodes is worn about the torso of a pregnant patient, the at least one of maternal or fetal biopotential signals detected by a plurality of electrode pairs selected from the plurality of electrodes of the wearable device, wherein a first electrode of each of the plurality of electrode pairs is a measurement electrode and a second electrode of each of the plurality of electrode pairs is a reference electrode;
select, by the processing circuitry and based on processing the at least one of maternal or fetal biopotential signals detected by the plurality of electrode pairs, one of the plurality of electrodes to use as the reference electrode during the monitoring session; and
control, by the processing circuitry, the plurality of electrodes to detect the at least one of maternal or fetal biopotential signals using the selected one of the plurality of electrodes as the reference electrode in each of the plurality of electrode pairs.

13. The method of claim 12, wherein the wearable device further comprises a wearable band configured to be worn about the torso of the patient, wherein the plurality of electrodes are affixed to or embedded in the wearable band.

14. The method of claim 12, further comprising supplying, via a driven electrode, a biasing voltage to a body of the patient.

15. The method of claim 14, further comprising:
selecting, based on processing the at least one of maternal or fetal biopotential signals detected by one or more of the plurality of electrode pairs, one of the plurality of electrodes to use as the driven electrode during the monitoring session.

16. The method of claim 12, wherein the wearable device further includes at least one of a temperature sensor or an accelerometer.

17. The method of claim 12, wherein one or more of the plurality of electrodes comprises a dry electrode.

18. The method of claim 12, further comprising:
automatically selecting, during the monitoring session and based on one or more parameters of the detected at least one of maternal or fetal biopotential signals, a different one of the plurality of electrodes to use as the reference electrode during the monitoring session; and
controlling the plurality of electrodes to detect the at least one of maternal or fetal biopotential signals using the selected different one of the plurality of electrodes as the reference electrode in each of the plurality of electrode pairs.

19. The method of claim 12, wherein the at least one of maternal or fetal biopotential signals include one or more of maternal electrocardiography signals, fetal electrocardiography signals, or maternal electromyography signals.

20. A non-transitory computer readable medium comprising instructions that when executed by processing circuitry cause the processing circuitry to:
receive, during a monitoring session during which a wearable device including a plurality of electrodes is worn about the torso of a pregnant patient, at least one of maternal or fetal biopotential signals detected by a plurality of electrode pairs selected from the plurality of electrodes of the wearable device, wherein a first electrode of each of the plurality of electrode pairs is a measurement electrode and a second electrode of each of the plurality of electrode pairs is a reference electrode;
select, based on processing the at least one of maternal or fetal biopotential signals detected by the plurality of electrode pairs of the wearable device, one of the plurality of electrodes to use as the reference electrode during the monitoring session; and
control the plurality of electrodes to detect the at least one of maternal or fetal biopotential signals using the selected one of the plurality of electrodes as the reference electrode in each of the plurality of electrode pairs.
